(19) United States
(12) Patent Application Publication
(10) Pub. No.: US 2026/0294640 A1
(43) Pub. Date: Oct. 1, 2026

(54) SPINAL IMPLANT DEVICE

(71) Applicant: Spineology Inc., St. Paul, MN (US)

(72) Inventors: Kyle Wolff, St. Paul Park, MN (US); John Liebelt, Lonsdale, MN (US); Aaron Weinard, Minnetonka, MN (US); Ian Malinowski, Henderson, MN (US)

(21) Appl. No.: 19/699,323

(22) Filed: Jun. 5, 2026

Related U.S. Application Data
(63) Continuation-in-part of application No. 19/309,285, filed on Aug. 25, 2025.
Application No. 19/309,285 is a continuation-in-part of application No. PCT/US2024/036184, filed on Jun. 28, 2024.
(60) Provisional application No. 63/523,902, filed on Jun. 28, 2023.

(51) Int. Cl. A61F 2/44; A61F 2/30

(52) U.S. Cl. CPC A61F 2/441; A61F 2/442; A61F 2002/30581; A61F 2002/3093

(57) ABSTRACT

A spinal implant is provided that is configured to receive a fill material. The spinal implant includes a wall defined by a plurality of strands braided together. The wall defines an interior chamber that extends between an anterior end and a posterior end. The wall has an interior side, an exterior side, and a wall thickness that extends between the interior side and the exterior side. The wall has a porosity that permits fluid passage through the wall thickness. The spinal implant is disposable in a collapsed state and in a filled state. The plurality of strands are braided in a manner that gives the interior chamber a volumetrically increasing configuration in a direction from the posterior end to the anterior end when the spinal implant is disposed in the filled state.

### CROSS-REFERENCE TO RELATED APPLICATIONS

This application is a continuation-in-part of U.S. patent application Ser. No. 19/309,285 filed Aug. 25, 2025, which claims priority to PCT/US2024/036184 filed Jun. 28, 2024, which claims priority to U.S. Patent Appln. No. 63/523,902 filed Jun. 28, 2023, all of which are hereby incorporated by reference in their entirety.

### BACKGROUND OF THE INVENTION

### 1. Technical Field

The present invention generally relates to implantable devices used in orthopedic surgeries, and more particularly to devices and methods used in spinal interbody fusion procedures.

### 2. Background Information

A spinal disc consists of three parts: a nucleus pulposis (the “nucleus”) which is a central portion that functions as a compression-resisting cushion, an annulus fibrosis (the “annulus”) which is a peripheral portion that functions as a tension-resisting hoop, and third the cartilaginous end plates (the “end plates”) that form the superior and inferior borders of the disc, consisting of the upper and lower surfaces of the vertebral body bones adjacent to the disc. Many studies have concluded that mechanical back pain is the most common and costly musculoskeletal condition affecting aging humans in modern societies. Mechanical back pain may be caused by several factors. Evidence suggests that degeneration of the spinal intervertebral disc is the most common condition causing stenosis, radiculopathy, and instability, any of which may lead to symptoms such as back and radiating limb pain.

Many devices have been invented for the purpose of easing the pain associated with degenerative disc disease. There is, however, a continuing need to provide improved devices adapted for treatment of degenerative disc disease.

### SUMMARY

According to an aspect of the present disclosure, a spinal implant is provided that is configured to receive a fill material. The spinal implant comprises a wall defined by a plurality of strands braided together. The wall defines an interior chamber that extends between an anterior end and a posterior end. The wall has an interior side, an exterior side, and a wall thickness that extends between the interior side and the exterior side. The wall has a porosity that permits fluid passage through the wall thickness and permits blood vessels and fibrous tissue to extend through the wall thickness. The spinal implant is disposable in a collapsed state and in a filled state. The plurality of strands are braided in a manner that gives the interior chamber a volumetrically increasing configuration in a direction from the posterior end to the anterior end when the spinal implant is disposed in the filled state.

In any of the aspects or embodiments described above and herein, the spinal implant may comprise a first lateral side, a second lateral side, a superior side, an inferior side, a length that extends between the posterior end and the anterior end, a width that extends between the first lateral side and the second lateral side, and a height that extends between the superior side and the inferior side.

In any of the aspects or embodiments described above and herein, in the filled state the height at the anterior end may be greater than the height at the posterior end.

In any of the aspects or embodiments described above and herein, in the filled state the width may be constant.

In any of the aspects or embodiments described above and herein, in the filled state the width at a first lengthwise point may be equal to a first width, the width at a second lengthwise point may be equal to a second width, and the second width may be greater than the first width.

In any of the aspects or embodiments described above and herein, in the filled state the height may increase at a constant rate in the direction from the posterior end to the anterior end.

In any of the aspects or embodiments described above and herein, in the filled state in a lengthwise direction the superior side and the inferior side may be separated from one another by a segmentation angle.

In any of the aspects or embodiments described above and herein, in the filled state, the superior side and the inferior side within a first lengthwise section of the implant contiguous with the posterior end may be separated from one another by a first segmentation angle, and the superior side and the inferior side within a second lengthwise section of the implant contiguous with the anterior end may be separated from one another by a second segmentation angle, and the first segmentation angle may be different from the second segmentation angle.

In any of the aspects or embodiments described above and herein, the wall defined by the plurality of strands braided together may have a braided configuration and the braided configuration may be uniform throughout the implant.

In any of the aspects or embodiments described above and herein, the wall defined by the plurality of strands braided together in a first section of the implant may have a first braided configuration and the wall defined by the plurality of strands braided together in a second section of the implant may have a second braided configuration.

In any of the aspects or embodiments described above and herein, the first section may have a first height that extends between the superior side and the inferior side, and the second section may have a second height that extends between the superior side and the inferior side, and the second height may be greater than the first height.

In any of the aspects or embodiments described above and herein, the first braided configuration may have a first number of strands and the second braided configuration may have a second number of strands, and the second number of strands may be greater than the first number of strands.

In any of the aspects or embodiments described above and herein, the first braided configuration may have a first pick count and the second braided configuration may have a second pick count, and the second pick count may be greater than the first pick count.

According to an aspect of the present disclosure, a spinal implant is provided that is configured to receive a fill material. The spinal implant comprises a unitary body that has a wall, and a length that extends between a posterior end and an anterior end. The wall has an interior side, an exterior side, and a wall thickness that extends between the interior side and the exterior side. The plurality of apertures extend through the wall, and the apertures are configured to permit fluid passage through the wall thickness. The wall defines an interior chamber that extends between the anterior end and the posterior end. The spinal implant is disposable in a collapsed state and in a normal state, and the spinal implant is configured to elastically transform from the collapsed state to the normal state. In the normal state, the interior chamber has a volumetrically increasing configuration from the posterior end to the anterior end.

In any of the aspects or embodiments described above and herein, the plurality of apertures may be produced by a machining process.

In any of the aspects or embodiments described above and herein, the spinal implant may comprise a first lateral side, a second lateral side, a superior side, an inferior side, a length that extends between the posterior end and the anterior end, a width that extends between the first lateral side and the second lateral side, and a height that extends between the superior side and the inferior side.

In any of the aspects or embodiments described above and herein, in the normal state the height at the anterior end may be greater than the height at the posterior end.

In any of the aspects or embodiments described above and herein, in the normal state the width at a first lengthwise point may be equal to a first width, the width at a second lengthwise point may be equal to a second width, and the second width may be greater than the first width.

In any of the aspects or embodiments described above and herein, the spinal implant may comprise a first structural band disposed adjacent the posterior end and a second structural band disposed adjacent the anterior end. The second structural band may be circumferentially longer than the first structural band.

In any of the aspects or embodiments described above and herein, the unitary body may comprise or consist of nitinol.

The foregoing features and elements may be combined in various combinations without exclusivity, unless expressly indicated otherwise. For example, aspects and/or embodiments of the present disclosure may include any one or more of the individual features or elements disclosed above and/or below alone or in any combination thereof. These features and elements as well as the operation thereof will become more apparent in light of the following description and the accompanying drawings. The following description and drawings are intended to be exemplary in nature and non-limiting.

### BRIEF DESCRIPTION OF THE DRAWINGS

FIG. 1 is a diagrammatic representation of a superior vertebra, an inferior vertebra, and a disc disposed therebetween.

FIG. 2 is a diagrammatic perspective view of a present disclosure spinal implant embodiment.

FIG. 3 is a diagrammatic side view of a present disclosure spinal implant embodiment.

FIG. 4 is a diagrammatic sectional view of the spinal implant embodiment shown in FIG. 3 along sectional line 4-4.

FIG. 5 is a diagrammatic representation of a superior vertebra, an inferior vertebra, and a disc disposed therebetween with a present disclosure multi-chamber spinal implant embodiment disposed in a cavity in the disc, with the implant shown in a collapsed state.

FIG. 6 is a diagrammatic representation of a superior vertebra, an inferior vertebra, and a disc as shown in FIG. 6 with the present disclosure multi-chamber spinal implant embodiment disposed in the disc cavity in an expanded state.

FIG. 7 is a diagrammatic representation of a Jersey knitting stitch.

FIG. 8 is a diagrammatic perspective view of a present disclosure spinal implant embodiment.

FIG. 8A is a diagrammatic sectional view of the spinal implant embodiment shown in FIG. 8 along sectional line 8A-8A.

FIG. 9 diagrammatically illustrates a side view of a present disclosure spinal implant embodiments.

FIG. 9A is a diagrammatic sectional view of the spinal implant embodiment shown in FIG. 9 along sectional line 9A-9A.

FIG. 9B is a diagrammatic sectional view of the spinal implant embodiment shown in FIG. 9 along sectional line 9B-9B.

FIG. 9C is a diagrammatic sectional view of the spinal implant embodiment shown in FIG. 9 along sectional line 9C-9C.

FIG. 10 is a diagrammatic perspective view of a present disclosure spinal implant embodiment.

FIG. 11 is a diagrammatic perspective view of a present disclosure spinal implant embodiment.

### DETAILED DESCRIPTION

The present disclosure is directed to a spinal implant device and system that may be utilized in an interbody fusion procedure and a method for using the same. As will be disclosed in greater detail herein, present disclosure spinal implant configurations may be configured in a plurality of different states; e.g., in a collapsed state, an unfilled state, a filled state, a fill expanded state, or in a normal state. The ability of the spinal implant to be in a collapsed state facilitates its use within an interbody fusion procedure, including use within a percutaneous interbody fusion procedure. The present disclosure is not limited to use within any particular interbody fusion procedure. The spinal implant may be transitioned from a collapsed state to a fill expanded state by inserting a fill material into an interior chamber of the implant. The present disclosure spinal implant is understood to provide a clinician with substantially greater ability to produce a desired orientation between adjacent spinal vertebrae and the benefits attendant therewith.

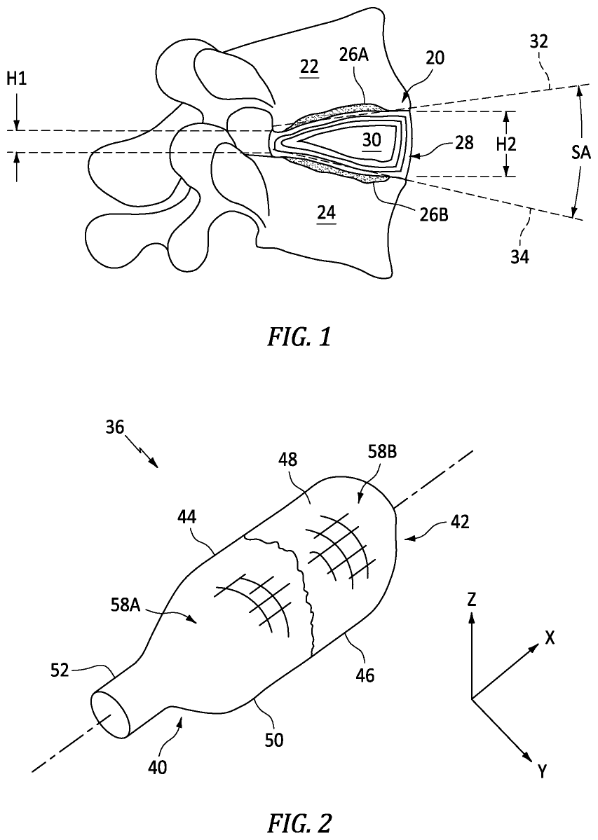

FIG. 1 diagrammatically illustrates a disc 20 disposed between a superior vertebra 22 and an inferior vertebra 24. The disc 20 is sectionally illustrated to show the inferior endplate 26A of the superior vertebra 22, the superior endplate 26B of the inferior vertebra 24, the annulus 28, and the nucleus 30. FIG. 1 includes a first plane 32 that is representative of the inferior endplate 26A of the superior vertebra 22 and a second plane 34 that is representative of the superior endplate 26B of the inferior vertebra 24. The included angle between the first and second planes 32, 34 is referred to hereinafter as the segmentation angle (“SA”). It should be noted that the lordosis angle of a spinal region (e.g., the lumbar region or the cervical region) is understood to be a measure of a collective curvature of a respective spinal region. It is further understood that there are other measurements of lordosis angle aspects (e.g., segmental lordosis, cumulative lordosis, and the like) that are sometimes used to refer to aspects of a lordosis angle. For sake of clarity, the term “segmentation angle” (which may contribute to the lordosis angle) is used herein to refer to the angle “SA” disposed within the sagittal plane defined by a first and second planes 32, 34. FIG. 1 also includes a disc height value “H1” disposed adjacent the posterior end of the disc 20 and a disc height value “H2” disposed adjacent the anterior end of the disc 20. The difference in disc height (H2>H1) and the segmentation angle “SA” illustrates the normal asymmetry of the disc 20, giving the disc 20 a “trapezoid-like” or “wedge-shaped” cross-sectional geometry.

Aspects of the present disclosure are directed to a plurality of spinal implant configurations. As will be detailed herein, in some embodiments a present disclosure spinal implant may be formed from a mesh of strands; e.g., a knitted mesh, a braided mesh, or the like. In some embodiments, a present disclosure spinal implant may be formed as a shaped unitary body comprising a metallic material or a polymer-metal composite material.

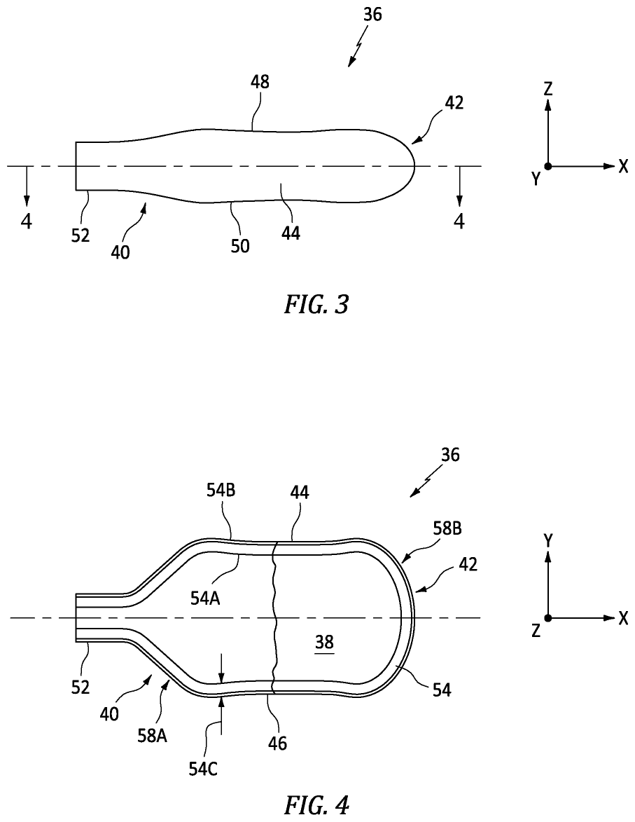

FIG. 2 is a diagrammatic perspective illustration of an unfilled present disclosure spinal implant 36 embodiment. FIG. 3 is a diagrammatic side view of the implant 36 embodiment like that shown in FIG. 2. FIG. 4 is a diagrammatic sectional view of the implant 36 embodiment shown in FIG. 3 along the sectional line 4-4.

Referring to FIG. 4, the spinal implant 36 is a unitary structure that includes an interior chamber 38 and may be described as having a posterior end 40, an anterior end 42, a first lateral side 44, a second lateral side 46, a superior side 48, and an inferior side 50. In some embodiments (e.g., like that shown in FIG. 2), the spinal implant 36 may include a fill port 52 that provides a passage into the interior chamber 38 of the implant 36. The present disclosure implants 36 do not require a fill port 52, however. To facilitate the description herein, the spinal implant 36 will be described herein as having a length (e.g., X-axis) that extends between the posterior end 40 and the anterior end 42, a width (e.g., Y-axis) that extends between the first lateral side 44 and the second lateral side 46, and a height (e.g., Z-axis) that extends between the superior side 48 and the inferior side 50.

The fill port 52 (if included) may be disposed in an open configuration and in a closed configuration. In the open configuration, the fill port 52 allows an instrument (e.g., a tube—not shown) to be inserted into the interior chamber 38 of the implant 36. In the closed configuration, the fill port 52 does not allow the escape of fill material from the interior chamber 38 via the fill port 52. In some instances, the spinal implant 36 may default to a closed configuration when the tube is withdrawn from the fill port 52; i.e., no further action is required to achieve the closed configuration. In other embodiments, the fill port 52 may be placed in a closed configuration by a suturing process, or by an adhesive, or by welding, or the like. The present disclosure is not limited to any particular fill port 52 configuration or fill port 52 closing procedure. As stated above, in some embodiments a fill port 52 may not be included. For example, the implant wall may be configured to allow a fill device (not shown; e.g., a catheter, a needle, a fill tube, or the like) to insert fill material through the wall 54 and into the interior chamber 38. Once the interior chamber 38 is filled and the fill device is removed, the wall 54 returns to its original form that prevents fill material to pass therethrough.

Referring to FIG. 4, the interior chamber 38 of the implant 36 is defined by a continuous wall 54 or by a plurality of walls 54 that collectively define the interior chamber 38. In those embodiments wherein the interior chamber 38 is defined by a single continuous wall 54, the interior chamber 38 may be described as having wall segments that are portions of the single continuous wall 54. The wall 54 includes an interior side 54A, an exterior side 54B, and a thickness 54C that extends between the opposing interior and exterior surfaces 54A, 54B.

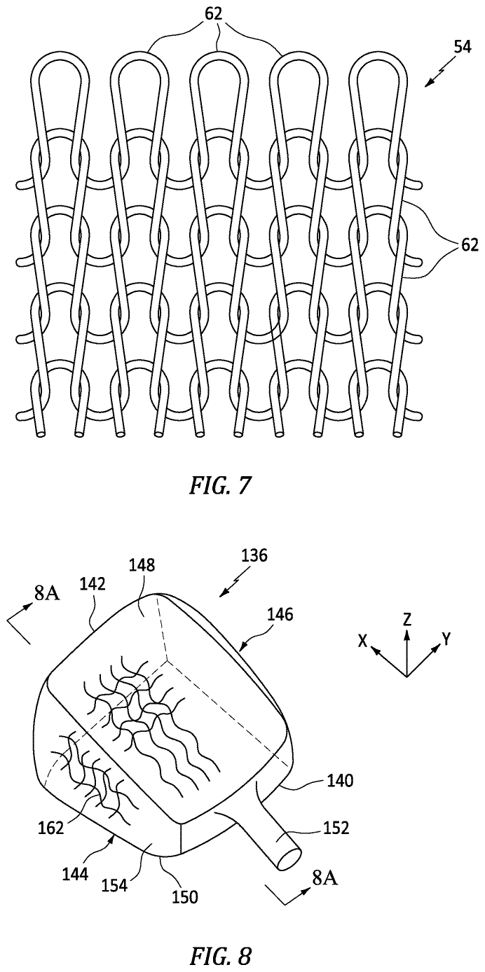

In those present disclosure implant embodiments that are formed from a mesh of strands (e.g., a knitted mesh, a braided mesh, or the like), the wall 54 of the present disclosure spinal implant 36 is both porous and pliable. The porosity of the wall 54 is chosen to permit the passage of fluids and solutions through the wall 54 while also preventing fill material 56 (see FIG. 6) from passing through the wall 54. The passage of fluids and solutions into and out of the interior chamber 38 is understood to facilitate the ingrowth, on-growth, and through-growth of blood vessels and fibrous tissue and bony trabeculae and consequently promote fusion between a fill material 56 (See FIG. 6) disposed within the interior chamber 38 and the vertebral endplates 26A, 26B. The size (i.e., cross-sectional area) of the pores (e.g., voids between adjacent strands 62) that create the porous nature of the wall 54 may be chosen based on the fill material 56 used within the spinal implant 36; e.g., may be based on the particulate size(s) within the fill material 56. The size of the pores within a wall 54 of the implant 36 may be uniform throughout the entirety of the respective wall 54, but that is not required. In some embodiments, the entirety of the implant wall 54 is porous. In some embodiments, less than all of the wall 54 is porous.

Referring to FIG. 7, in some embodiments, the wall 54 of the present disclosure spinal implant 36 may be configured as a mesh of strands 62; i.e., an interlaced arrangement of strands 62. As will be detailed herein, a non-limiting example of an interlaced arrangement of strands is a knitting stitch; e.g., a Jersey knitting stitch as diagrammatically shown in FIG. 7. Another example of an interlaced arrangement of strands 62 is one in which the strands 62 are disposed in a braided arrangement.

The strands 62 may comprise biocompatible materials. For example, a strand material used for medical sutures may be used to form the mesh. Other non-limiting examples of acceptable mesh strand materials include titanium and other biocompatible metals in various material configurations, nitinol, biodegradable materials, polymeric materials, and others. In some embodiments, mesh strands may be treated (e.g., by coating, absorbance, or the like) with a bioactive solution such as one containing an antibiotic, a pharmaceutical agent, osteoconductive material, or a bone morphogenic protein such as, for example, recombinant human bone morphogenetic protein (rhBMP), and any combinations thereof. U.S. Patent Publication No. 2008/0113008, entitled “Absorbent Fabric Implant”, which is hereby incorporated by reference in its entirety, discloses examples of bioactive materials that may be utilized with the present disclosure spinal implant 36. The present disclosure is not limited to any particular mesh strand materials, other than one that is suitable for the application at hand.

As indicated herein, in some embodiments the spinal implant 36 may be disposed in a collapsed state, an unfilled state, or a fill expanded state. In a collapsed state, the volume of the implant interior chamber 38 may be zero (or nearly zero); e.g., the interior sides 54A of the wall 54 (or wall segments) that define the interior chamber 38 are substantially in contact with one another thereby producing a zero volume interior chamber 38. In an unfilled state, the interior sides 54A of the wall 54 are spaced apart from one another and collectively define an “at rest” interior chamber 38 of the spinal implant 36. In the unfilled state, the wall 54 of the spinal implant 36 is at rest; i.e., the wall 54 is not under tension and no force is acting on the wall 54 that would cause the wall 54 to elongate or expand (i.e., deviate from its at rest configuration) and consequently cause the interior chamber 38 to volumetrically increase in size. The term “fill expanded state” as used herein refers to an implant 36 configuration wherein the interior chamber 38 is at a volume resulting from a pressure being applied to the interior side 54A of the wall 54. For example, and as detailed herein, if sufficient fill material 56 is packed into the interior chamber 38, the fill material 56 will produce a pressure force acting on the interior side 54A of the implant wall 54. That pressure force may (as will be described herein) cause portions of the interior chamber 38 to increase volumetrically.

In some embodiments, the interior chamber 38 of the present disclosure implant 36 includes at least a first chamber segment 58A and a second chamber segment 58B, with each chamber segment 58A, 58B having a different wall configuration. The term “wall configuration” as used herein refers to the physical characteristics of the wall 54 itself and does not refer to the geometry in which the wall 54 is disposed. Hence, a first wall and a second wall may have the same geometry and still have different wall configurations. As will be described in detail herein, as a result of the respective wall configurations, one of the chamber segments 58A, 58B is configured to volumetrically expand a greater percentage than the other chamber segment 58B, 58A when the walls 54 of each chamber segment 58A, 58B are subjected to the same pressure force; a pressure force acting on the walls from the interior chamber 38. In this manner, the size of one chamber segment 58A, 58B may be increased disproportionately relative to another chamber segment 58B, 58A during the filling process, which may facilitate establishing a desired separation (“distraction”) between inferior and superior vertebra 22, 24 and a desired segmentation angle therebetween.

Referring to FIGS. 2-4, to facilitate the description herein, the first chamber segment 58A may be referred to as a posterior chamber segment that is disposed adjacent the posterior end 40 of the spinal implant 36 and the second chamber segment 58B may be referred to as an anterior chamber segment disposed adjacent the anterior end 42 of the spinal implant 36. To be clear, the present disclosure implant 36 is not limited to an embodiment having a posterior chamber segment and an anterior chamber segment. The chamber segments 58A, 58B (e.g., the posterior and anterior chamber segments) may be open to one another; i.e., no barrier is disposed between the posterior and anterior chamber segments that would inhibit the passage of fill material therebetween. The posterior chamber segment may be described as having a first wall configuration and the anterior chamber segment may be described as having a second wall configuration, wherein the second wall configuration is different than the first wall configuration. In some embodiments, the plurality of chamber segments 58A, 58B may each have the same unfilled state volume; e.g., both the posterior chamber segment and the anterior chamber segment have an unfilled state volume equal to 50% of the total interior chamber unfilled state volume. In some embodiments, the chamber segments may have unequal unfilled state volumes; e.g., the posterior chamber segment may have an unfilled state volume equal to 33% of the total interior chamber unfilled state volume, and the anterior chamber segment may have an unfilled state volume equal to 67% of the total interior chamber unfilled state volume. The present disclosure is not limited to any particular volumetric ratio between the posterior chamber segment and the anterior chamber segment.

In those embodiments that include a wall 54 configured as a mesh of strands (e.g., a knitted wall configuration), the wall 54 may include mesh arrangements that allow one chamber segment 58A, 58B to volumetrically expand a greater percentage than another chamber segment 58B, 58A when the interior side of each chamber segment is subjected to the same pressure force. The “same pressure force” assumes that the fill material 56 packed into the chamber segments 58A, 58B is substantially uniformly packed throughout the interior chamber 38 thereby resulting in substantially the same pressure force being applied to the wall 54 of the spinal implant 36 throughout the chamber segments 58A, 58B. Hence, differences in volumetric expansion are not a result of non-uniform fill material packing. A “volumetric expansion” for a given chamber segment 58A, 58B may occur via the mesh expanding, or the mesh strands elongating, or the like, or any combination thereof, as a result of the pressure force applied by the fill material 56.

The volumetric expansion of an implant chamber segment 58A, 58B between an unfilled state and a fill expanded state may be described in terms of a volumetric expansion ratio. The volumetric expansion ratio (VER) may be defined as follows:

$$V E R = \frac{Chamber Segment Volume in Fill Expanded State}{Chamber Segment Volume in Unfilled State}$$

If a chamber segment 58A, 58B does not expand at all from the chamber segment volume in an unfilled state to the chamber segment volume in a fill expanded state, the VER would have a value of one (1.0). A VER value of 1.1 would mean that the chamber segment volume in a fill expanded state is 10% greater than the chamber segment volume in an unfilled state, a VER value of 1.25 would mean that the chamber segment volume in a fill expanded state is 25% greater than the chamber segment volume in an unfilled state, and so on.

Different mesh strand patterns (i.e., different wall configurations) may have different expansion characteristics (expansion characteristics are sometimes referred to as “extension” characteristics). As an example, a Jersey knitting stitch may extend (e.g., stretch) more when under tension than an interlocking knitting stitch made from the same strand material. Hence, different stitch patterns (i.e., different wall configurations) may result in different volumetric expansion characteristics. Volumetric expansion for a given chamber segment 58A, 58B may also occur by other wall configuration means; e.g., mesh elongation, inclusion of different material strands, mesh directional orientation, and the like.

There are several ways volumetric expansion ratio (VER) differences between chamber segments 58A, 58B can be accomplished in a spinal implant 36. For example, a first VER may be produced by using a first mesh arrangement (i.e., a first wall configuration) within a first chamber segment 58A and using a second mesh arrangement (i.e., a second wall configuration) within a second chamber segment 58B. The first mesh arrangement may be achieved using a first Jersey stitch configuration and the second mesh arrangement may be achieved using a second Jersey stitch configuration that is different from the first Jersey stitch configuration. The specific differences between the first and second Jersey stitch configurations may lie in stitch parameters. For example, the tallness (or the “height”) of the stitch courses along a length of the mesh arrangement may be varied between the first and second Jersey stitch configurations. As another example, the needles per course may be varied between the first and second Jersey stitch configurations. In this example, the second mesh arrangement of the second chamber segment 58B may be configured for greater volumetric expansion than the first mesh arrangement of the first chamber segment 58A. The present disclosure is not limited to these example stitch parameter variations within a given stitch type. Another example of creating VER differences between chamber segments 58A, 58B, includes using a first stitch type in a first chamber segment and a second stitch type in a second chamber segment, wherein the first stitch type is different than the second stitch type. For example, a first stitch type (e.g., an interlock stitch) may be used in a first chamber segment 58A and a second stitch type (e.g., a Jersey stitch) may be used in a second chamber segment 58B. In this example, the expansion characteristic of the interlock stitch may be less than the expansion characteristic of the Jersey stitch. Hence, for a given uniform amount of pressure applied to the first chamber segment 58A (comprising the interlock stitch), the first chamber segment 58A will expand less than the second chamber segment 58B (comprising the Jersey stitch). The present disclosure is not limited to any particular means for creating volumetric expansion ratio (VER) differences between chamber segments in an implant 36.

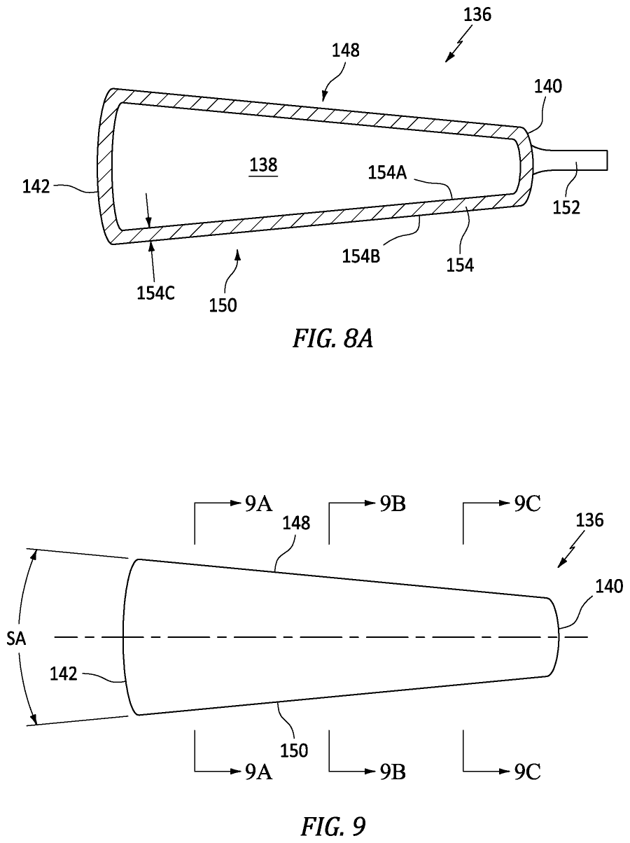

Referring to FIGS. 8 and 8A, in some present disclosure embodiments the wall 154 of the present disclosure spinal implant 136 may be configured as a plurality of strands 162 braided together to create a braided mesh of strands 162; i.e., an interlaced braided arrangement of strands 162. As indicated herein, the strands 162 may comprise biocompatible materials, including but not limited to, a strand material used for medical sutures, titanium strands, nitinol strands, biodegradable materials, polymeric strands, and the like.

The braided embodiment of the spinal implant 136 may be configured in a plurality of different states; e.g., in a collapsed state, an unfilled state, or a filled state.

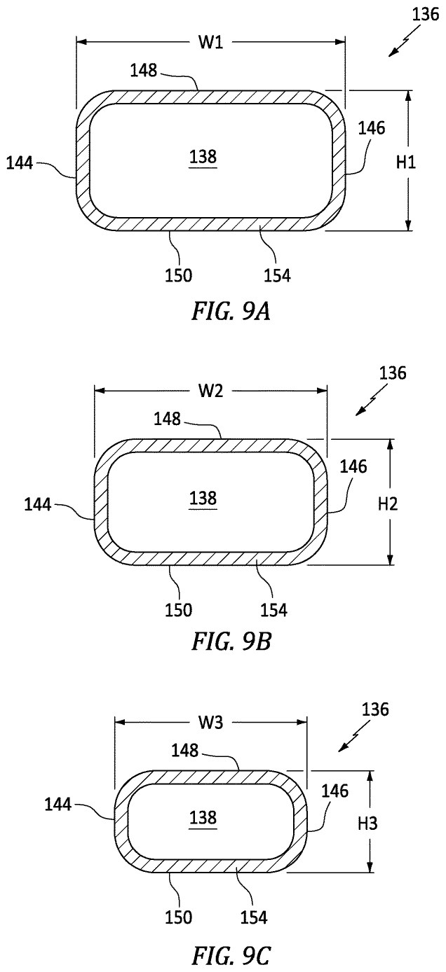

The braided embodiment of the spinal implant 136 is a unitary structure that includes an interior chamber 138 defined by the wall 154 of the implant 136. The implant 136 may be described as having a posterior end 140, an anterior end 142, a first lateral side 144, a second lateral side 146, a superior side 148, and an inferior side 150. In some embodiments, the spinal implant 136 may include a fill port 152 (not required; shown in FIGS. 8-8A, not illustrated in FIGS. 9-11) that provides a passage into the interior chamber 138 of the implant 136. The fill port 152 may be configured as described herein and shown in FIG. 4. As detailed herein, the spinal implant 136 will be described herein as having a length (e.g., X-axis) that extends between the posterior end 140 and the anterior end 142, a width (e.g., Y-axis) that extends between the first lateral side 144 and the second lateral side 146, and a height (e.g., Z-axis) that extends between the superior side 148 and the inferior side 150. In similar manner, the interior chamber 138 may be described herein as having a length (e.g., X-axis) that extends between the interior wall surfaces of the posterior and anterior ends 140, 142, a width (e.g., Y-axis) that extends between the interior wall surfaces of the first and second lateral sides 144, 146, and a height (e.g., Z-axis) that extends between the interior wall surfaces of the superior and inferior sides 148, 150.

Referring to FIG. 8A, in the braided embodiment of the spinal implant 136, the interior chamber 138 of the implant 136 is defined by a continuous wall 154 or by a plurality of walls 154 that collectively define the interior chamber 138. In those embodiments wherein the interior chamber 138 is defined by a single continuous wall 154, the interior chamber 138 may be described as having wall segments that are portions of the single continuous wall 154. The wall 154 includes an interior side 154A, an exterior side 154B, and a thickness 154C that extends between the opposing interior and exterior surfaces 154A, 154B.

The porosity of the wall 154 is chosen to permit the passage of fluids and solutions through the wall 154 while also preventing fill material 56 (see FIG. 6) from passing through the wall 154. As indicated herein, the passage of fluids and solutions into and out of the interior chamber 138 is understood to facilitate the ingrowth, on-growth, and through-growth of blood vessels and fibrous tissue and bony trabeculae and consequently promote fusion between a fill material 56 disposed within the interior chamber 138 and the vertebral endplates 26A, 26B (e.g., see FIG. 6). The size (i.e., cross-sectional area) of the pores (i.e., voids defined by adjacent strands 162) that create the porous nature of the wall 154 may be chosen based on the fill material 56 used within the spinal implant 136. As indicated herein, the size of the pores within a wall 154 of the implant 136 may be uniform throughout the entirety of the respective wall 154, but that is not required.

Present disclosure implant embodiments (including the braided implant 136 shown diagrammatically in FIG. 8) may be configured to have an interior chamber 138 that increases in volume in the direction from the posterior end 140 to the anterior end 142. FIG. 9 diagrammatically illustrates a side view of an implant 136 with sectional lines 9A-9A, 9B-9B, and 9C-9C. The sectional line 9A-9A is closest to the anterior end 142 of the implant 136, the sectional line 9C-9C is closest to the posterior end 140 of the implant 136, and the sectional line 9B-9B is disposed between sectional lines 9A-9A and 9C-9C. FIG. 9A diagrammatically illustrates a cross-section of the implant 136 at sectional line A-A. The height of the implant 136 (i.e., the distance between the superior side 148 and the inferior side 150) at cross-section 9A-9A is “H1” and the width of the implant 136 (i.e., the distance between the first lateral side 144 and the second lateral side 146) at cross-section 9A-9A is “W1”. FIG. 9B diagrammatically illustrates a cross-section of the implant 136 at sectional line 9B-9B. The height of the implant 136 at cross-section 9B-9B is “H2” and the width of the implant 136 at cross-section 9B-9B is “W2”. The height of the implant 136 at cross-section 9C-9C is “H3” and the width of the implant 136 at cross-section 9C-9C is “W3”. In this diagrammatic example, the implant 136 has a wedge configuration that expands between the posterior and anterior ends 140, 142. The height of the implant 136 (and the interior chamber 138) at cross-section 9A-9A is greater than the height at cross-section 9B-9B, and the height of the implant 136 (and the interior chamber 138) at cross-section 9B-9B is greater than the height at cross-section 9C-9C; i.e., H1>H2>H3. The cross-sectional area (Area_{9A}) of the implant 136 (and its interior chamber 138) at the 9A-9A cross-section is greater than the cross-sectional area (Area_{9B}) at the 9B-9B cross-section, and the cross-sectional area at the 9B-9B cross-section is greater than the cross-sectional area (Area_{9C}) at the 9C-9C cross-section; i.e., Area_{9A}>Area_{9B}>Area_{9C}. The width of the implant 136 (and the interior chamber 138) from the posterior end 140 to the anterior end 142 may be constant (e.g., W1=W2=W3) but that is not required. In some embodiments, the implant 136 (and the interior chamber 138) may have a first width in a first lengthwise section and a different width in a second lengthwise section; e.g., W1>W3. Differences in width may be part of the volumetric increase in the direction from the posterior end 140 to the anterior end 142. As can be seen in FIG. 9, the wedge shape may be described as having a segmentation angle (“SA”) extending between the inferior side 150 of the implant 136 and the superior side 148 of the implant 136, with the angle opening in the direction from the posterior end 140 to the anterior end 142.

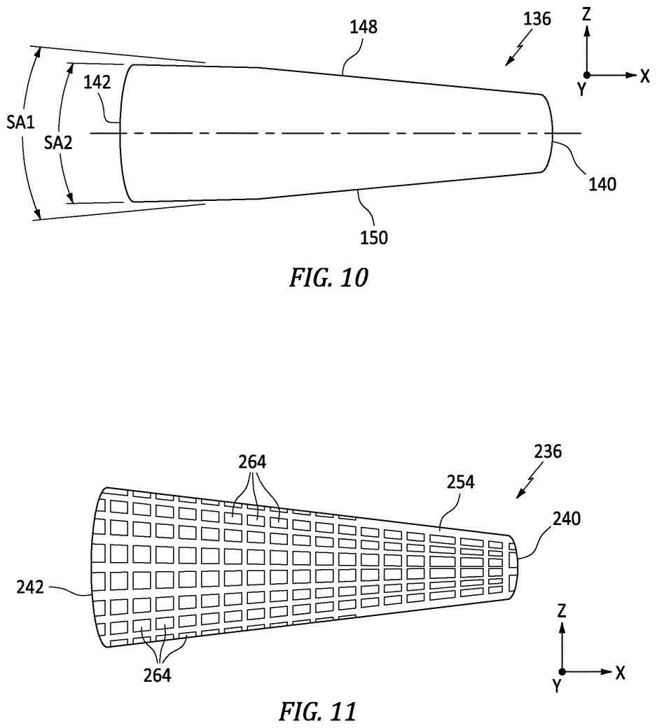

The wedge shape of the implant 136 diagrammatically shown in FIGS. 9-9B is provided to illustrate the posterior-to-anterior increasing volume configuration of the implant 136, and the implant 136 is not limited to this configuration. In the embodiment shown in FIG. 9, the inferior and superior sides 150, 148 of the implant 136 are diagrammatically shown as planar segment segments; e.g., the segmentation angle is constant from the posterior end 140 to the anterior end 142. In some embodiments, the segmentation angle may vary in lengthwise segments. FIG. 10 illustrates an example of an implant 136 that is configured with a posterior-to-anterior increasing volume configuration of the implant 136 that has a first section extending along a first segmentation angle (SA1) and a second section extending along a second segmentation angle (SA2), wherein the second segmentation angle is different from the first segmentation angle (SA1≠SA2; e.g., SA1>SA2). The present disclosure contemplates that the implant 136 may have different segmentation angles to provide improved implant 136—disc cavity conformance.

The posterior-to-anterior increasing volume configuration of the braided implant 136 may be accomplished by changing the braiding technique, or the settings of the machine performing the braiding, or the strand density, or the like, or any combination thereof. Braiding techniques that may be used to accomplish the posterior-to-anterior increasing volume configuration include, but are not limited to, varying the braiding angle of the strands 162 within the braid, and/or varying the braiding pick count (sometimes referred to as “picks per inch”, “PPI”, or “pick count”) of the strands 162 within the braid. For example, a higher pick count may be used in the region of the implant 136 at the posterior end 140, and the pick count decreased (e.g., gradually or step wise) in the direction toward the anterior end 142. A higher pick count may be used to produce a denser, less flexible braided structure and a lower pick count may be used to produce a more open, flexible braided structure. Another braiding technique that may be used to accomplish the posterior-to-anterior increasing volume configuration is a change in the number of strands 162 within the braided implant 136. For example, the number of strands 162 within the braided implant 136 may increase (e.g., step wise) in the posterior to anterior direction. The additional strands 162 are understood to facilitate the volumetric increase of the implant 136. A braiding technique may be used to create a braided configuration. An implant 136 that is formed using a single braiding technique may be described as having uniform braided configuration. An implant 136 having a first section that is formed using a first braiding technique and a second section that is formed using a second braiding technique may be described as having a first braided configuration (formed using the first braiding technique) and a second braided configuration (formed using the second braiding technique) and so on for each different braiding technique.

In some embodiments, a wedge shaped mandrel may be used during the production of a present disclosure braided implant 136 with a posterior-to-anterior increasing volume configuration.

As indicated herein, in a collapsed state the volume of the braided implant interior chamber 138 may be zero (or nearly zero). In a filled state (i.e., the braided implant 136 filled with fill material), the pressure being applied to the wall 154 by the fill material is typically uniform. The braided implant 136 may be configured to not substantially expand in the filled state beyond the volume of the unfilled state of the braided implant 136, or the braided implant 136 may be configured to uniformly expand to some degree when in the filled state, or sections of the braided implant 136 may expand (increase volumetrically) a greater amount than other sections of the braided implant 136 when in the filled state; e.g., a section of the implant 136 adjacent the anterior end 142 may expand more than a section of the implant 136 adjacent the posterior end 140 when filled.

Referring to FIG. 11, some present disclosure spinal implant 236 embodiments may comprise a metallic material or a polymer-metal composite material. In such embodiments, the implant 236 has one or more walls 254 and may be shaped as a unitary body having a posterior-to-anterior increasing volume configuration with an interior chamber as detailed herein. The shaped implant 236 is configured to be disposed in a collapsed configuration or in a normal configuration. The normal configuration is the geometric configuration of the shaped implant 236 when the implant 236 is at-rest without any constraints limiting the geometric expansion of the implant 236. In the collapsed configuration, the shaped implant 236 is disposed in a geometrically constrained form that may be inserted into a disk cavity via a cannula as may be used in a minimally invasive surgery. The shaped implant 236 is configured to elastically transform from the collapsed configuration to the normal configuration once the geometric constraints are removed from the shaped implant 236; i.e., the geometric transformation occurs when the shaped implant 236 is disposed within the surgically created disk cavity.

The wall(s) 254 of the shaped implant 236 are configured as a porous structure having a plurality of apertures 264 sized to permit the passage of fluids and solutions through the wall 254 while also preventing fill material from passing through the wall 254. The porosity may be created by a machining process such as laser-cutting, electro-discharge machining (“EDM”), grinding or the like. The wall porosity may be uniform throughout the implant or it may differ in different regions of the implant 236. In some embodiments, the shaped implant 236 may include structural elements (e.g., circumferential bands) that differ in circumferential length to create the posterior-to-anterior increasing volume configuration; e.g., a first circumferential length (CL1) at a first position adjacent the posterior end 240, a third circumferential length (CL3) at a third position adjacent the anterior end 242, and a second circumferential length (CL2) at a second position disposed lengthwise between the first and third positions, where CL1>CL2>CL3. In some instances, a spinal implant 236 comprising a metallic material or a polymer-metal composite material may be formed using an additive manufacturing process.

Nitinol is a particularly useful metallic material for a shaped spinal implant 236 embodiment as described above. Nitinol is known to have a “recovery” property that allows a structure like the shaped implant 236 to be disposed in a collapsed configuration that facilitates insertion via minimally invasive surgery, and to elastically transform from the collapsed configuration to the normal configuration once the shaped implant 236 is disposed within the surgically created disk cavity. The “recovered” normal configuration within the disk cavity is understood to facilitate the filling process and specifically the creation of a vertebral support structure having a desirable geometry; e.g., a wedge shaped geometry.

The fill material 56 used to fill the implant 36, 136, 236 may include one or more of the following, or any other biocompatible material judged to have the desired physiologic response, or any combination thereof: demineralized bone material, morselized bone graft, cortical, cancellous, or cortico-cancellous, including autograft, allograft, or xenograft; any bone graft substitute or combination of bone graft substitutes, or combinations of bone graft and bone graft substitutes, or bone inducing substances, including but not limited to: calcium phosphates, calcium sulfates, calcium carbonates, hydroxyapatite, bone morphogenic proteins, calcified and/or decalcified bone derivatives; bone cements, such as injectable ceramic and polymethylmethacrylate bone cements, or any osteoconductive biocompatible material known to promote bone formation, titanium and other biocompatible metals in various material configurations, resorbable metals such as magnesium (Mg) and zinc (Zn), and polymeric particles including but not limited to polyether ether ketone (PEEK), polyether ketone ketone (PEKK), polyethylene (PE), polyurethane (PU), polycarbonate polyurethane (PCU), polylactic acid (PLA), polyamide (PA), poly lactic-co-glycolic acid (PLGA), and polyglycolide acid (PGA). U.S. Pat. No. 7,959,683, “Packed Demineralized Cancellous Tissue Forms for Disc Nucleus Augmentation, Restoration, or Replacement and Methods of Implantation”, which is hereby incorporated by reference in its entirety, discloses examples of fill material 56 that may be used with the present invention. The present disclosure is not limited to any particular type of fill material 56.

The present disclosure spinal implant 36, 136, 236 embodiments are understood to provide an improved capability to produce a desired orientation between a superior vertebra 22 and an inferior vertebra 24; e.g., vertebrae separation distance (“distraction”) and/or segmentation angle. The variable expansion between implant chamber segments 58A, 58B and the posterior-to-anterior increasing volume configuration are understood to give the surgeon performing the procedure a greater ability to customize the spinal implant 36 to create the desired vertebral orientation; e.g., vertebrae separation distance and segmentation angle. In addition, the configuration of the present disclosure spinal implants 36, 136, 236 (i.e., customizable chamber segments 58A, 58B, segmentation angles, and the like) is understood to allow a surgeon to choose a spinal implant 36, 136, 236 having a chamber segment volumetric expansion ratio or a posterior-to-anterior increasing volume configuration that best suits the procedure at hand. The present disclosure spinal implant 36, 136, 236 embodiments are also understood to provide an improvement in conformity between the spinal implant 36, 136, 236 and the respective endplate 26A, 26B. The improved conformity is understood to facilitate the amount of fusion between the endplates and the spinal implant 36, 136, 236; e.g., by increasing the amount of area in contact between the spinal implant 36, 136, 236 and the respective endplate 26A, 26B.

As indicated herein, present disclosure spinal implant 36, 136, 236 embodiments may be utilized in an interbody fusion procedure to alleviate issues associated with a medical condition such as degenerative disc disease. The procedure that may be used to insert the spinal implant 36, 136, 236 may be chosen based on numerous factors including which spinal vertebrae are to be fused, the reason for the spinal fusion, and the general health and body shape of the patient. Some traditional interbody fusion procedures (“open surgery”) require direct visualization by the clinician and may require bone to be cut and significant retraction of soft tissue and nerve roots. Alternatively, it may be possible to perform a minimally invasive form of interbody fusion often referred to as percutaneous spinal interbody fusion. The present disclosure spinal implant 36, 136, 236 embodiments may be used in either of these types of interbody fusion procedures (and others), and as will be detailed herein can provide significant clinical benefit to the patient. To facilitate the description herein, an example of how the present disclosure spinal implant 36, 136, 236 may be used in a percutaneous spinal interbody fusion is provided.

Percutaneous interbody fusion is performed under indirect visualization using x-ray or other imaging and/or navigation technologies, including robotics and endoscopy. Because neural tissue cannot be seen on x-ray, active neural monitoring may be used to avoid nerve damage that may otherwise occur during the procedure. There are two types of neural monitoring that are generally used in spine surgery: electromyography (EMG) and somatosensory evoked potential (SSEP). When using neural monitoring in the spine, the surgeon evaluates nerve potential by checking for evoked responses. An instrument, such as a neural stimulating component, may be used to mechanically manipulate or electrically stimulate the nerve in order to evoke a response.

Dilators may be used with the neural monitoring to incrementally establish an acceptable approach path and cannula for the surgical instruments used in the interbody fusion. The dilation step includes penetrating the surface of the annulus portion of the target disc. The orientation of the approach path relative to the patient's spine (e.g., an anterior approach, a posterior approach, a lateral approach, a posterolateral approach, an anterolateral approach, and so on) may be chosen by the clinician based on the circumstances of the patient. It is understood that a variety of different approaches may be used in a percutaneous interbody fusion using a present disclosure spinal implant 36, 136, 236. Once the acceptable approach path and cannula are established and the annulus portion of the target disc 20 is penetrated, a cavity is created in the nucleus of the disc 20 by removing at least a portion of disc nucleus. The disc removal process may include liberating disc nucleus material (e.g., by cutting and scraping) using a tool having one or more blades. The liberated disc nucleus material may be removed using a rongeur or the like. The liberation and removal of disc material creates the cavity. The geometry of the cavity may be chosen in view of the procedure at hand and the geometry of the desired implant. The disc cavity preparation process may include preparing a fusion bed of bleeding bone at the vertebral endplates to facilitate new bone growth for fusion to occur. U.S. Pat. No. 8,906,094, “System and Method for Performing Percutaneous Spinal Interbody Fusion”, which is hereby incorporated by reference in its entirety, discloses examples of a minimally invasive disc preparation methodology that may be used with the present invention. The present disclosure is not limited to any particular disc cavity preparation process. Once the cavity and the endplate fusion beds are established, the present disclosure spinal implant 36, 136, 236 in a collapsed state may be inserted percutaneously.

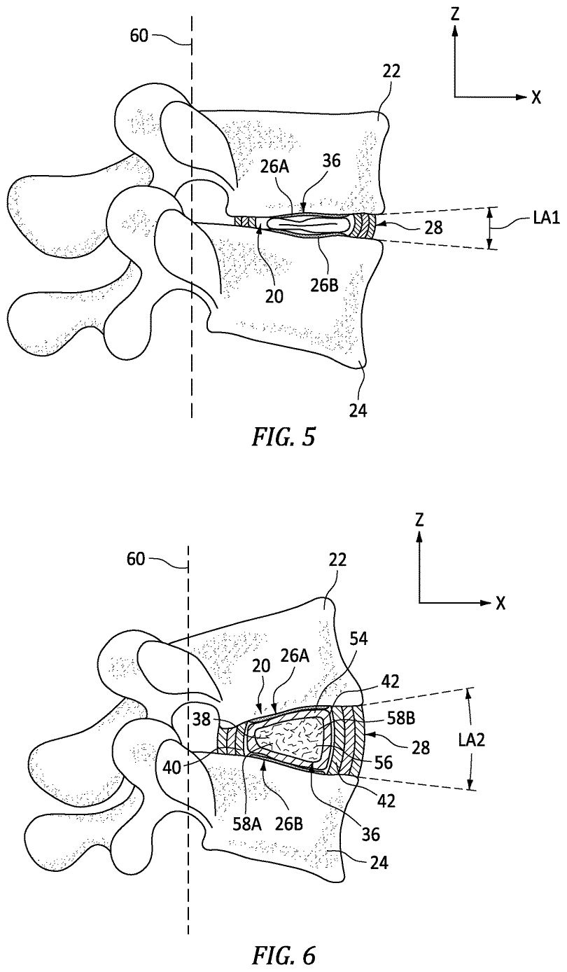

FIG. 5 diagrammatically illustrates a pair of spinal vertebrae (e.g., a superior vertebra 22 and an inferior vertebra 24) with a disc 20 disposed therebetween. In this view, the disc 20 is shown diagrammatically sectioned to facilitate the view and the description of the end plates 26A, 26B, the annulus 28, a cavity where the disc nucleus (not shown) resided prior to being excised, and a present disclosure spinal implant 36. In FIG. 5, the implant 36 (or implant 136, 236) is inserted into the cavity (e.g., via a cannula) in a collapsed state and the inferior endplate 26A of the superior vertebra 22 is almost parallel to the superior endplate 26B of the inferior vertebra 24. Hence, the segmentation angle (“LA1”) between the superior vertebra 22 and the inferior vertebra 24 is minimal. The dashed vertical line 60 extending through the vertebra 22, 24 is representative of the coronal plane.

FIG. 6 diagrammatically illustrates the superior and inferior spinal vertebra 22, 24, the disc 20, and the spinal implant 36 shown in FIG. 5. In FIG. 6, the implant 36 is shown in a fill expanded state with the posterior chamber segment (i.e., first chamber segment 58A) and the anterior chamber segment (i.e., second chamber segment 58B) packed with a fill material 56. With the implant 36 in the fill expanded state, the height between the superior vertebra 22 and the inferior vertebra 24 (i.e., the distraction) has increased in both the posterior region and the anterior region, and the segmentation angle between the endplates 26A, 26B of the superior vertebra 22 and the inferior vertebra 24 has increased from “LA1” to “LA2” (LA2>LA1) relative to the diagrammatic view shown in FIG. 5. Here again, the dashed vertical line 60 extending through the vertebra 22, 24 is representative of the coronal plane.

In this example, the present disclosure spinal implant 36 may be configured so that one of the posterior chamber segment or the anterior chamber segment has a volumetric expansion ratio (VER) that is greater than the VER of the other of the posterior chamber segment or the anterior chamber segment. The VER of the posterior chamber segment and the VER of the anterior chamber segment of the interior chamber 38 of the spinal implant 36 may be chosen to create a desired orientation between the superior and inferior vertebra 24 in terms of vertebrae separation distance and intervertebral angle within the sagittal plane; e.g., the segmentation angle. In similar fashion, an implant 136, 236 may be chosen having a particular posterior-to-anterior increasing volume configuration to create a desired orientation between the superior and inferior vertebra 22, 24 in terms of vertebrae separation distance and intervertebral angle within the sagittal plane. As indicated herein, other embodiments of the present disclosure implants 36, 136, 236 may be configured to create a desired orientation between the superior and inferior vertebra 22, 24 relative to the coronal and axial planes, or any combination of the sagittal, coronal, and axial planes.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

The terms “substantially,” “about,” “approximately,” and other similar terms of approximation used throughout this patent application are intended to encompass variations or ranges that are reasonable and customary in the relevant field. These terms should be construed as allowing for variations that do not alter the basic essence or functionality of the invention. Such variations may include, but are not limited to, variations due to manufacturing tolerances, materials used, or inherent characteristics of the elements described in the claims and should be understood as falling within the scope of the claims unless explicitly stated otherwise. As an example, the present application describes that the term “same pressure force” assumes that the fill material packed into the chamber segments is “substantially” uniformly packed throughout the interior chamber 38 thereby resulting in substantially the same pressure force being applied to the wall 54 of the spinal implant 36 throughout the chamber segments. In this example, the term “substantially” is used to indicate that although the fill material packing may not be exactly uniform, it is packed sufficiently uniform such that the variations create inconsequential variations in the application of pressure force.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible.

What is claimed:

1. A spinal implant configured to receive a fill material, comprising:
a wall defined by a plurality of strands braided together, wherein the wall defines an interior chamber that extends between an anterior end and a posterior end, wherein the wall has an interior side, an exterior side, and a wall thickness that extends between the interior side and the exterior side, wherein the wall has a porosity that permits fluid passage through the wall thickness and permits blood vessels and fibrous tissue to extend through the wall thickness; and
wherein the spinal implant is disposable in a collapsed state and in a filled state; and
wherein the plurality of strands are braided in a manner that gives the interior chamber a volumetrically increasing configuration in a direction from the posterior end to the anterior end when the spinal implant is disposed in the filled state.

2. The spinal implant of claim 1, wherein the spinal implant further comprises a first lateral side, a second lateral side, a superior side, an inferior side, a length that extends between the posterior end and the anterior end, a width that extends between the first lateral side and the second lateral side, and a height that extends between the superior side and the inferior side.

3. The spinal implant of claim 2, wherein in the filled state the height at the anterior end is greater than the height at the posterior end.

4. The spinal implant of claim 3, wherein in the filled state the width is constant.

5. The spinal implant of claim 3, wherein in the filled state the width at a first lengthwise point is equal to a first width, the width at a second lengthwise point is equal to a second width, and the second width is greater than the first width.

6. The spinal implant of claim 2, wherein in the filled state the height increases at a constant rate in the direction from the posterior end to the anterior end.

7. The spinal implant of claim 2, wherein in the filled state in a lengthwise direction the superior side and the inferior side are separated from one another by a segmentation angle.

8. The spinal implant of claim 2, wherein in the filled state, the superior side and the inferior side within a first lengthwise section of the implant contiguous with the posterior end are separated from one another by a first segmentation angle, and the superior side and the inferior side within a second lengthwise section of the implant contiguous with the anterior end are separated from one another by a second segmentation angle, and the first segmentation angle is different from the second segmentation angle.

9. The spinal implant of claim 1, wherein the wall defined by the plurality of strands braided together has a braided configuration and the braided configuration is uniform throughout the implant.

10. The spinal implant of claim 1, wherein the wall defined by the plurality of strands braided together in a first section of the implant has a first braided configuration and the wall defined by the plurality of strands braided together in a second section of the implant has a second braided configuration.

11. The spinal implant of claim 10, wherein the spinal implant further comprises a superior side and an inferior side; and
wherein the first section has a first height that extends between the superior side and the inferior side, and the second section has a second height that extends between the superior side and the inferior side, and the second height is greater than the first height.

12. The spinal implant of claim 10, wherein the first braided configuration has a first number of strands and the second braided configuration has a second number of strands, and the second number of strands is greater than the first number of strands.

13. The spinal implant of claim 10, wherein the first braided configuration has a first pick count and the second braided configuration has a second pick count, and the second pick count is greater than the first pick count.

14. A spinal implant configured to receive a fill material, comprising:
a unitary body having a wall, and a length that extends between a posterior end and an anterior end;
wherein the wall has an interior side, an exterior side, and a wall thickness that extends between the interior side and the exterior side; and
wherein plurality of apertures extend through the wall, and the apertures are configured to permit fluid passage through the wall thickness;
wherein the wall defines an interior chamber that extends between the anterior end and the posterior end; and
wherein the spinal implant is disposable in a collapsed state and in a normal state, and the spinal implant is configured to elastically transform from the collapsed state to the normal state; and
wherein in the normal state, the interior chamber has a volumetrically increasing configuration from the posterior end to the anterior end.

15. The spinal implant of claim 14, wherein the plurality of apertures are produced by a machining process.

16. The spinal implant of claim 14, wherein the spinal implant further comprises a first lateral side, a second lateral side, a superior side, an inferior side, a length that extends between the posterior end and the anterior end, a width that extends between the first lateral side and the second lateral side, and a height that extends between the superior side and the inferior side.

17. The spinal implant of claim 16, wherein in the normal state the height at the anterior end is greater than the height at the posterior end.

18. The spinal implant of claim 16, wherein in the normal state the width at a first lengthwise point is equal to a first width, the width at a second lengthwise point is equal to a second width, and the second width is greater than the first width.

19. The spinal implant of claim 14, further comprising a first structural band disposed adjacent the posterior end and a second structural band disposed adjacent the anterior end;
wherein the second structural band is circumferentially longer than the first structural band.

20. The spinal implant of claim 14, wherein the unitary body comprises nitinol.
